(19) United States
(12) Patent Application Publication
(10) Pub. No.: US 2018/0256562 A1
(43) Pub. Date: Sep. 13, 2018

(54) Transdermal Delivery System

(71) Applicant: Amneal Pharmaceuticals LLC, Bridgewater, NJ (US)

(72) Inventors: Sameer Sachdeva, Piscataway, NJ (US); Tarun Goswami, Edison, NJ (US); Jay Audett, Somerset, NJ (US)

(21) Appl. No.: 15/758,269

(22) PCT Filed: Sep. 9, 2016
(86) PCT No.: PCT/US16/50904

§ 371 Date: Mar. 7, 2018

Related U.S. Application Data
(60) Provisional application No. 62/218,124, filed on Sep. 14, 2015.

(51) Int. Cl. A61K 31/485; A61K 9/00; A61K 9/70; A61K 47/12; A61K 47/14; A61K 47/32; A61M 35/00; A61F 13/02; A61F 13/00

(52) U.S. Cl. CPC A61K 31/485; A61K 9/0014; A61K 9/7061; A61K 9/7069; A61K 47/12; A61K 47/14; A61F 2013/0296; A61M 35/00; A61F 13/0259; A61F 13/0289; A61F 13/0253; A61F 13/00063; A61M 2207/00; A61K 47/32

(57) ABSTRACT

Described is a transdermal device comprising a backing layer; a single layer adhesive matrix comprising buprenorphine or a salt thereof, a pressure sensitive adhesive including a silicone-type adhesive blended with an acrylate-type adhesive, a solubilizer, a permeation enhancer, and a crystallization inhibitor; and a release layer. Also described is a method of relieving pain and a method of preparing a transdermal delivery system.

### TECHNICAL FIELD

The present invention relates generally to the field of transdermal drug delivery. More particularly, the invention relates to a single-layer buprenorphine-based, silicone and acrylate pressure sensitive adhesive formulation, and its use in making devices for improved transdermal delivery of buprenorphine.

### BACKGROUND

Transdermal drug delivery has been accepted as a potential non-invasive route of drug administration, with advantages of prolonged therapeutic action, decreased side effects, easy use, and better patient compliance. Pressure sensitive adhesive matrix patches are known and typically include an inert, impervious backing layer, a pressure sensitive adhesive layer containing the drug and optional selected excipients, and a release liner that is peeled off and discarded before applying the patch to the skin. Transdermal drug delivery patches dispense a drug at a controlled rate by presenting the drug for absorption in an efficient manner, for example, with a minimal residual drug or degradation of the drug, and prevent complications from failure of a patient to comply with a therapeutic regimen.

Buprenorphine (5R,6R,7R,9R,13S,14S)-17-cyclopropylmethyl-7-[(S)-3,3-dimethyl-2-hydroxybutan-2-yl]-6-methoxy-4,5-epoxy-6,14-ethanomorphinan-3-ol) is a semi-synthetic opioid derivative of thebaine. It is an opioid partial agonist that is used to treat opioid addiction, to control acute pain, and to control chronic pain. Buprenorphine is available in a variety of formulations, e.g. salt forms, sublingual tablets, solutions, transdermal preparations.

Butrans®, supplied by Purdue Pharma L.P., is a transdermal system providing systemic delivery of buprenorphine continuously for 7 days. Five different strengths of Butrans® are available: 5, 7.5, 10, 15, and 20 mcg/hour. The proportion of buprenorphine mixed in the adhesive matrix is the same in each of the 5 strengths. Butrans® is a rectangular or square patch consisting of a protective liner and functional layers. FIG. 1 illustrates the Butrans® transdermal system. As illustrated in FIG. 1, proceeding from the outer surface of the Butrans® transdermal system 100 toward the surface adhering to the skin, the layers are a web-backing layer 110; an adhesive rim without buprenorphine 120; a separating layer 130 over the buprenorphine-containing adhesive matrix 140; and a peel-off release liner 150. Before use, the release liner 150 covering the buprenorphine-containing adhesive matrix 140 and the adhesive rim without buprenorphine 120 is removed and discarded. The active ingredient in Butrans® is buprenorphine, and the inactive ingredients are levulinic acid, oleyl oleate, polyvinyl pyrrolidone (povidone), and polyacrylate cross-linked with aluminum.

WO2014/195352 to Lts. Lohmann Therapie-Systeme AG describes a transdermal patch for the transdermal administration of buprenorphine comprising a self-adhesive layered structure comprising a buprenorphine-impermeable backing layer, and a buprenorphine-containing pressure-sensitive adhesive layer on the backing layer. The adhesive layer comprises a pressure-sensitive adhesive based on polysiloxane, an analgesically effective amount of buprenorphine base, soluble polyvinylpyrrolidone, and levulinic acid.

Butrans® delivers buprenorphine to the skin to treat patients in pain for a time period of 7 days and allows, in a fixed dosing regimen, a once-weekly patch change, which is beneficial in terms of patient compliance. The long administration periods of Butrans®, however, may cause problems with skin irritation, particularly because the size of the transdermal patch is relatively large. Additionally, a large amount of excess drug in the transdermal patch of Butrans® is costly and can lead to illicit use. The transdermal system of WO2014/195352 does not have good adhesion properties and has stability concerns.

There remains a need for methods of treating patients with buprenorphine that provide effective analgesic levels of buprenorphine for prolonged periods of time, while eliminating or minimizing dependence, tolerance, and side effects, thus providing a safe and effective method of pain management. More specifically, there is an on-going need to provide transdermal drug delivery systems which are smaller in size, have reduced drug loading, and have an acceptable buprenorphine skin flux.

### SUMMARY

A first aspect of the invention pertains to a transdermal delivery system. In a first embodiment, a transdermal delivery system comprises: a backing layer; an adhesive matrix comprising buprenorphine or a salt thereof, a pressure sensitive adhesive including a silicone-type adhesive blended with an acrylate-type adhesive, a solubilizer, a permeation enhancer, and a crystallization inhibitor; and a release liner.

In a second embodiment, the transdermal delivery system of the first embodiment is modified, wherein the silicone-type adhesive is selected from pressure sensitive adhesives comprising silicone polymer and resin, and the acrylate-type adhesive is selected from the group consisting of an acrylate-vinylacetate polymer.

In a third embodiment, the transdermal delivery system of the first and second embodiments is modified, wherein the solubilizer comprises a carboxylic acid.

In a fourth embodiment, the transdermal delivery system of the third embodiment is modified, wherein the carboxylic acid is a C₃ to C₂₄ carboxylic acid or a C₃ to C₂₄ keto acid.

In a fifth embodiment, the transdermal delivery system of the first through fourth embodiments is modified, wherein the solubilizer is selected from levulinic acid, lauric acid, lactic acid, oleic acid, linoleic acid or mixtures thereof.

In a sixth embodiment, the transdermal delivery system of the first through fifth embodiments is modified, wherein the permeation enhancer comprises a fatty acid ester.

In a seventh embodiment, the transdermal delivery system of the sixth embodiment is modified, wherein the fatty acid ester is a glycerol ester of a C₆ to C₁₈ fatty acids.

In an eighth embodiment, the transdermal delivery system of the sixth and seventh embodiments is modified, wherein the fatty acid ester is a monoglyceride, diglyceride, triglyceride, or combinations thereof.

In a ninth embodiment, the transdermal delivery system of the first through eighth embodiments is modified, wherein the permeation enhancer is selected from oleyl oleate, glyceryl monooleate, lauryl lactate, or mixtures thereof.

In a tenth embodiment, the transdermal delivery system of the first through ninth embodiments is modified, wherein the crystallization inhibitor is selected from one or more of cross-linked polyvinylpyrrolidone, polyvinylpyrrolidone (PVP), or hydroxypropyl methylcellulose (HPMC), and other cellulosic based inhibitors.

In an eleventh embodiment, the transdermal delivery system of the first through tenth embodiments is modified, wherein the backing layer is impermeable to the buprenorphine.

In a twelfth embodiment, the transdermal delivery system of the first through eleventh embodiments is modified, wherein the backing layer comprises films of polyethylene, polyethylene terephthalate (PET), polypropylene, polyurethane, ethylene vinyl acetate (EVA), polyamide, metal foils, or paper, alone or coated with a polymeric material, a PVC foam or a woven or non-woven fabric.

In a thirteenth embodiment, the transdermal delivery system of the first through twelfth embodiments is modified, wherein the silicone-type adhesive and the acrylate-type adhesive are present in a ratio range of 1 to 1.8 of silicone-type adhesive to acrylate type adhesive.

In a fourteenth embodiment, the transdermal delivery system of the first through thirteenth embodiments is modified, wherein the release liner comprises one or more of paper, coated paper, plastic films, polyolefins made of high density polyethylene (HDPE), low density polyethylene (LDPE), polypropylene (PP) plastic resin, fluoropolymer-coated films.

In a fifteenth embodiment, the transdermal delivery system of the first through fourteenth embodiments is modified, wherein the adhesive matrix further comprises a solvent selected from ethanol, ethyl acetate, isopropyl alcohol, heptanes, and mixtures thereof.

A second aspect of the present invention is directed to a transdermal delivery system. In a sixteenth embodiment, a transdermal delivery system comprises: a backing layer; an adhesive matrix comprising: 3 to 15 w/w buprenorphine, 20 to 60% w/w silicone adhesive, 20 to 60% w/w polyacrylate adhesive, 3 to 15% w/w cross-linked polyvinylpyrrolidone, 3 to 15% w/w carboxylic acid, 3 to 15% w/w fatty acid; and a release liner.

In a seventeenth embodiment, the transdermal delivery system of the sixteenth embodiment is modified, wherein the adhesive matrix further comprises a solvent selected from ethanol, ethyl acetate, isopropyl alcohol, heptanes and mixtures thereof.

In an eighteenth embodiment, the transdermal delivery system of the first through seventeenth embodiments is modified, wherein the adhesive matrix forms a single layer on the backing layer, and wherein there is no peripheral layer.

A third aspect of the present invention is directed to a method of relieving pain. In nineteenth embodiment, a method of relieving pain comprises: applying to skin of a patient in need thereof the transdermal delivery system of the first through eighteenth embodiments.

In a twentieth embodiment, the method of the nineteenth embodiment is modified, wherein the transdermal delivery system is applied to the skin for a time period in a range of 1 to 7 days.

A fourth aspect of the present invention is direct to a method of preparing a transdermal delivery system. In a twenty-first embodiment, a method of preparing a transdermal delivery system comprises: dispersing a crystallization inhibitor in a first solvent to form a first solution; adding a second solvent to the first solution to form a second solution; adding a solubilizer and a permeation enhancer to the second solution to form a first mixture; adding a pressure sensitive adhesive including a silicone-type adhesive blended with an acrylate-type adhesive to the first mixture to form a second mixture; adding buprenorphine base or a salt thereof to the second mixture to form an adhesive matrix mixture; and coating the adhesive matrix mixture on a backing layer.

In a twenty-second embodiment, the method of the twenty-first embodiment is modified, wherein the crystallization inhibitor is selected from one or more of cross-linked polyvinylpyrrolidone, polyvinylpyrrolidone (PVP), or hydroxypropyl methylcellulose (HPMC), and other cellulosic based inhibitors

In a twenty-third embodiment, the method of the twenty-first and twenty-second embodiments is modified, wherein the first solvent and the second solvent are selected from ethanol, ethyl acetate isopropyl alcohol, heptanes, and mixtures thereof.

In a twenty-fourth embodiment, the method of the twenty-first through twenty-third embodiments is modified, wherein the solubilizer comprises a carboxylic acid.

In a twenty-fifth embodiment, the method of the twenty-fourth embodiment is modified, wherein the carboxylic acid is a C₃ to C₂₄ carboxylic acid or a C₃ to C₂₄ keto acid.

In a twenty-sixth embodiment, the method of the twenty-first through twenty-fifth embodiments is modified, wherein the solubilizer is selected from levulinic acid, lauric acid, lactic acid, oleic acid, or mixtures thereof.

In a twenty-seventh embodiment, the method of the twenty-first through twenty-sixth embodiments is modified, wherein the permeation enhancer comprises a fatty acid ester.

In a twenty-eighth embodiment, the method of the twenty-first through twenty-seventh embodiments is modified, wherein the fatty acid ester is a glycerol ester of a C₆ to C₁₈ fatty acid.

In a twenty-ninth embodiment, the method of the twenty-seventh and twenty-eighth embodiments is modified, wherein the fatty acid ester is a monoglyceride, diglyceride, triglyceride, or combinations thereof.

In a thirtieth embodiment, the method of the twenty-first through twenty-ninth embodiments is modified, wherein the permeation enhancer is selected from oleyl oleate, glyceryl monooleate, lauryl lactate, or mixtures thereof.

In a thirty-first embodiment, the method of the twenty-first through thirtieth embodiments is modified, wherein the backing layer comprises films of polyethylene, polyethylene terephthalate (PET), polypropylene, polyurethane, ethylene vinyl acetate (EVA), polyamide, metal foils, or paper, alone or coated with a polymeric material , a PVC foam or a woven or non-woven fabric.

In a thirty-second embodiment, the method of the twenty-first through thirty-first embodiments is modified, wherein the silicone-type adhesive and the acrylate-type adhesive are present in a ratio range of about 1 to about 1.8 of silicone-type adhesive to acrylate type adhesive.

In a thirty-third embodiment, the method of the twenty-first through thirty-second embodiments is modified, wherein the silicone-type adhesive is selected from the pressure sensitive adhesives comprising silicone polymer and resin, and the acrylate-type adhesive is selected from the group consisting of an acrylate-vinylacetate polymer.

A fourth aspect of the present invention is directed to an adhesive matrix. In a thirty-fourth embodiment, an adhesive matrix comprises: buprenorphine or a salt thereof; a pressure sensitive adhesive including a silicone-type adhesive blended with an acrylate-type adhesive; a solubilizer; a permeation enhancer; and a crystallization inhibitor.

In a thirty-fifth embodiment, the adhesive matrix of the thirty-fourth embodiment is modified, wherein the adhesive matrix is formulated into a single layer on a backing layer and wherein there is no peripheral layer.

### BRIEF DESCRIPTION OF THE DRAWINGS

The disclosure may be more completely understood in consideration of the following detailed description of various embodiments of the disclosure in connection with the accompanying drawings, in which:

FIG. 1 provides a cross-section view of transdermal patch according to the prior art;

FIG. 2 provides a cross-section view of an exemplary transdermal patch;

FIG. 3 provides a diffusion profile for a transdermal delivery system according to the Examples;

FIG. 4 provides a diffusion profile for a transdermal delivery system according to the Examples; and

FIG. 5A-5B provide microscopic images of a formulation prepared according to the examples, showing crystal growth for transdermal delivery systems after seeding with buprenorphine.

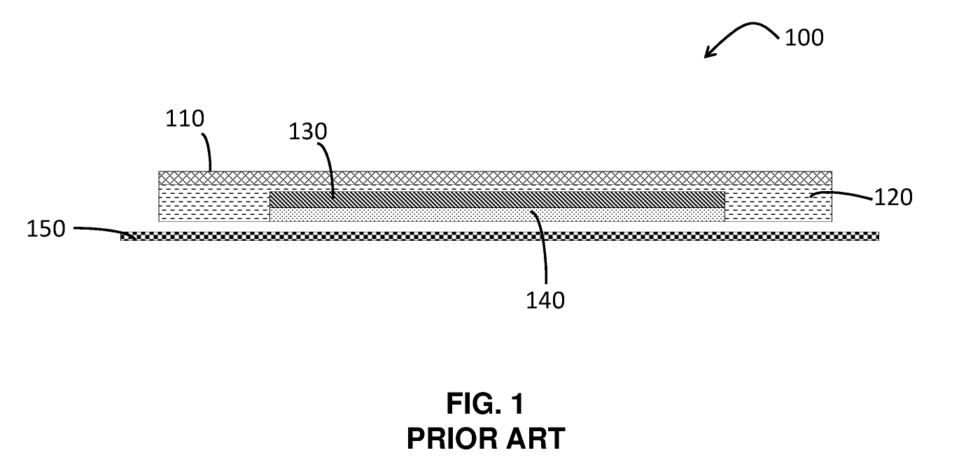

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Opioids are medications that relieve pain. Opioids reduce the intensity of pain signals reaching the brain and affect those brain areas controlling emotion, which diminishes the effects of a painful stimulus. Buprenorphine is a partially synthetic opiate that is more potent than other opiates (e.g. morphine), but has a much better safety profile. Buprenorphine has a low oral bioavailability and is considered by some to be habit forming. There is a need for a stable transdermal delivery system that can be used to treat opioid addiction in higher dosages, to control moderate acute pain in non-opioid-tolerant patients in lower dosages, and to control moderate chronic pain in even smaller doses.

Embodiments of the present invention are directed to a transdermal delivery system comprising a backing layer, an adhesive matrix comprising buprenorphine, and a release liner. Surprisingly, it was found that an adhesive matrix comprising buprenorphine or a salt thereof, a pressure sensitive adhesive including a silicone-type adhesive blended with an acrylate-type adhesive, a solubilizer, a permeation enhancer, and a crystallization inhibitor resulted in a transdermal delivery system having a reduced amount of buprenorphine and/or having a reduced surface area when compared to currently marketed transdermal delivery systems. Additionally, the transdermal delivery system of one or more embodiments exhibits higher skin flux as compared to the currently marketed product, Butrans®, with the same amount of buprenorphine loading and without the use of a peripheral layer. The transdermal delivery system of one or more embodiments may be used to deliver same or higher amounts of buprenorphine across the skin, as compared to commercially available product, Butrans®.

The transdermal delivery system of one or more embodiments is available as a matrix patch, and comprises an adhesive matrix containing buprenorphine between an impermeable backing layer and a release liner. The adhesive matrix system includes buprenorphine base or a salt thereof, a solubilizer, a permeation enhancer, a crystallization inhibitor, and a pressure sensitive adhesive. Due to higher skin flux, the size of the transdermal patch may be reduced when compared to the commercially marketed product.

With respect to the terms used in this disclosure, the following definitions are provided.

As used herein, the terms “transdermal patch,” “stable transdermal patch,” or “stable transdermal delivery system” are used interchangeably and refer to a medicated adhesive system, wherein effectively negligent crystallization is observed, which is placed on the skin to deliver a specific dose of medication through the skin and into the bloodstream. As recognized by one skilled in the art, because the skin is an effective barrier, only drugs whose molecules are small enough to penetrate the skin can be delivered by a transdermal patch.

As used herein, the term “matrix patch” refers to a transdermal patch which has an inert, impervious or impermeable backing layer, an active agent or drug for delivery, a semisolid adhesive matrix, and a release liner. The drug is incorporated into the adhesive matrix, such that the drug is located within the adhesive. A matrix patch does not contain an absorbent pad, which simplifies the manufacturing process.

Buprenorphine (5R,6R,7R,9R,13S,14S)-17-cyclopropylmethyl-7-[(S)-3,3-dimethyl-2-hydroxybutan-2-yl]-6-methoxy-4,5-epoxy-6,14-ethanomorphinan-3-ol) has the chemical structure of Formula (I):

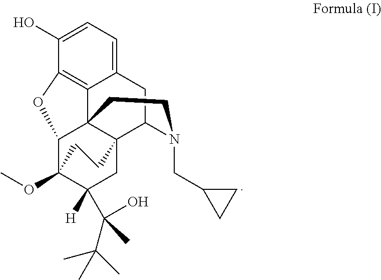

Buprenorphine is generally administered in the free base form. Pharmaceutically acceptable salts may be selected from those known in the art, such as the hydrochloride, sulphate, phosphate, tartrate, maleinate, oxalate, acetate, and lactate salts. In one or more specific embodiments, the pharmaceutically acceptable salt is a hydrochloride salt. The quantity of buprenorphine contained in the transdermal system of one or more embodiments is a quantity sufficient to provide a pharmaceutically or physiologically effective dosage rate of the active agent to a patient/host in need thereof. In one or more embodiments, the buprenorphine may be present in the formulation in an amount effective for chronic pain relief.

In one or more embodiments, the buprenorphine base or a salt thereof can be used in the transdermal delivery system in an amount in the range of about 3% to about 15% w/w (dry weight), including about 3% to about 10% w/w (dry weight), and about 5% to about 10% w/w (dry weight).

An analgesically effective amount of buprenorphine base or a pharmaceutically acceptable salt thereof may vary from about 1 mg to about 50 mg, including from about 2 mg to about 30 mg, and from about 2 mg to about 25 mg. In one or more embodiments, the transdermal delivery system contains from about 1 to about 35 mg buprenorphine base or a pharmaceutically acceptable salt thereof. As will be appreciated by one of skill in the art, the amount of buprenorphine base or a pharmaceutically acceptable salt thereof can be adjusted based upon the size of the transdermal patch and/or the indication of use.

In one or more embodiments, delivery rates of buprenorphine will usually be in the range of about 2 to about 250 mcg/hour, including the ranges of 2 to 100 mcg/hour, 2 to 75 mcg/hour, 2 to 50 mcg/hour, 2 to 25 mcg/hour, and 2 to 20 mcg/hour, including 5 mcg/hour, 7.5 mcg/hour, 10 mcg/hour, 15 mcg/hour and 20 mcg/hour.

As used herein, the term “skin flux” refers to the intrinsic flux of a drug/pharmaceutical active ingredient diffusing across a transdermal delivery system to the skin. In other words, skin flux is the amount of a drug/active pharmaceutical ingredient delivered by a transdermal delivery system to permeate the skin. As used herein, the phrase “acceptable skin flux” refers to an amount of drug in the range of about 2 to about 250 mcg/hour, including the ranges of 2 to 100 mcg/hour, 2 to 75 mcg/hour, 2 to 50 mcg/hour, 2 to 25 mcg/hour, and 2 to 20 mcg/hour, including 5 mcg/hour, 7.5 mcg/hour, 10 mcg/hour, 15 mcg/hour and 20 mcg/hour delivered by the transdermal delivery system to permeate the skin.

As used herein, the term “adhesive matrix” or “adhesive matrix layer” or “pressure sensitive adhesive layer” refers to a pressure sensitive adhesive layer, which serves to carry the active agent or the drug but also serves to attach the patch to the skin. The adhesive matrix includes a permeation enhancer, a crystallization inhibitor, a solubilizer and a pressure sensitive adhesive which is cast onto the material to be used as a non-releasable backing layer. The terms permeation enhancer, crystallization inhibitor and solubilizer are terms given their ordinary meaning as understood by one skilled in the art. The adhesive matrix includes a pressure sensitive adhesive which is cast onto the material to be used as a release liner or a backing layer.

In one or more embodiments, the adhesive matrix is made in such a manner that components of the adhesive and their solvents are mixed with the drug and/or other substances and then coated on a suitable sheet, intended to function as a disposable release liner, and the solvents are removed in a drying process. It is noted that the transdermal patch of one or more embodiments generally has a coating weight that is 95% the weight of currently marketed transdermal patches. Then, a non-releasable backing layer is applied over the adhesive matrix layer. The result is a web-like structure comprised of a pressure-sensitive adhesive matrix layer, containing the drug, sandwiched between a backing layer on one side and a disposable release liner on the other. The web can be cut into suitable sizes and shapes to produce pressure sensitive adhesive transdermal drug delivery patches.

In one or more embodiments, the transdermal patch has a size in the range of 3 to 50 cm², including a range of about 5 to 40 cm², a range of about 5 to about 30 cm², and a range of about 5 to about 25 cm².

As used herein, the terms “backing” or “backing sheet” or “backing layer” or “non-releasable backing layer” or “impermeable backing layer” refer to a layer or web applied over the adhesive matrix, which permits transfer of the drug to the wearer, but prevents transfer of the drug to non-wearers. The backing layer serves as a protective cover for the active agent, e.g. buprenorphine, and may also provide a support function. Examples of materials that may be suitable for use as backing layers in the present invention include films of high and low density polyethylene; polyesters such as poly(ethylene phthalate) and polyethylene terephthalate (PET); polypropylene; polyvinylchloride; polyurethane; ethylene vinyl acetate (EVA), polyamide; metal foils (e.g. aluminum foil); metal foil laminates of such suitable polymer films; textile fabrics; or paper, alone or coated with a polymeric material; a PVC foam; or a woven or non-woven fabric; or mixtures thereof. Illustrative wovens include KOB® 051, 053, and 055 woven polyesters (Karl Otto Braun.) Illustrative non-woven fabrics include polyesters.

Generally, there are no specific restrictions with regard to the thickness of the backing layer. In one or more embodiments, the backing layer can be any appropriate thickness which will provide the desired protective and support functions. In specific embodiments, the backing layer has a thickness in the range of from about 5 to about 200 microns, including a range of about 5 to about 100 microns, about 5 to about 50 microns, and about 5 to about 25 microns. In one or more embodiments, the backing layer is a PET-EVA laminate which is approximately 1 to 4 mils in thickness (1 mil=0.001 inch).

As used herein, the terms “release liner” or “release layer” refer to a film or sheet applied during the manufacture of the transdermal patch used to prevent the adhesive matrix from prematurely adhering to the skin. A release liner is a removable protective sheet that is removed before the patch is applied on the patient. A release liner is a removable protective sheet that has been rendered “non-stick” to the adhesive matrix. Generally, the release liner is in contact with the adhesive matrix and provides a convenient means of maintaining the integrity of the adhesive matrix until the desired time of application.

In one or more embodiments, the materials suitable for use as release liners include the materials used for the production of the backing layer. In other embodiments, examples of materials that may be suitable for use as release liners in the present invention include paper, allophane, polyvinyl chloride, coated paper, plastic films, polyolefins typically made of high density polyethylene (HDPE), low density polyethylene (LDPE), polypropylene (PP) plastic resin, fluoropolymer- coated films, silicone-coated films, and fluoropolymer-coated polyethylene terephthalate (PET) film.

Generally, there are no specific restrictions with regard to the thickness of the release liner. In one or more embodiments, the release liner can be any appropriate thickness. In specific embodiments, the release liner has a thickness in the range of from about 1 to about 200 microns, including a range of about 1 to about 100 microns, about 2 to about 100 microns, and about 1 to about 50 microns. In one or more embodiments, the release liner is about 1 to about 4 mils in thickness.

In one or more embodiments, the release liner may be the same size as the adhesive matrix layer and/or may be the same size as the backing layer. In other embodiments, the release liner may be oversized as compared with the adhesive matrix layer. Without intending to be bound by theory, it is thought that use of an oversized release liner may help prevent the adhesive matrix from becoming distorted or relaxing during the shipping and handling processes.

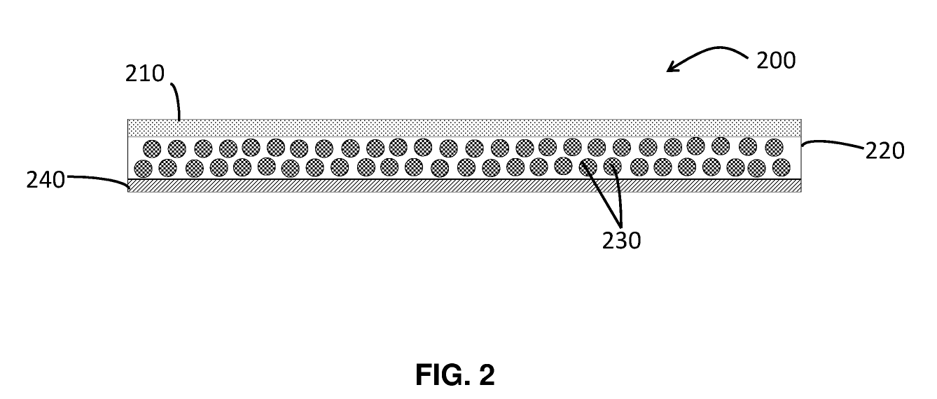

Referring to FIG. 2, a cross-section view of an exemplary transdermal matrix patch is provided. Matrix patch 200 has a backing layer 210 and an adhesive matrix 220 including particles of active agent 230, such as buprenorphine, is located on a release liner 40.

In one or more embodiments, buprenorphine base or a salt thereof as an active agent is dispersed in an adhesive matrix. In addition to buprenorphine, the adhesive matrix comprises a pressure sensitive adhesive comprising a silicone-type adhesive blended with an acrylate-type adhesive, a solubilizer, a permeation enhancer, and a crystallization inhibitor.

As used herein, the term “press-sensitive adhesive” refers to a material or combination of materials that provides suitable tack for quick bonding to various skin types, including wet or perspired skin, suitable adhesive and cohesive qualities, long lasting adhesion to the skin, a high degree of flexibility, a permeability to moisture, and compatibility to active ingredients, e.g. buprenorphine, and film-substrates. The use of pressure sensitive adhesives (PSAs) including a blend of polyacrylate adhesives and silicone adhesives, which are used to hold the patch to the skin, have desirable properties such as resistance to oxidation, permeability to water vapor and oxygen, good tack behavior and better shear strength; all of which are provided at a moderate cost.

In one or more embodiments, examples of a pressure sensitive adhesive that may be suitable for use in the adhesive matrix include a silicone type adhesive blended with an acrylate type adhesive. More specifically, suitable silicone adhesives include pressure sensitive adhesives made from silicone polymer and resin. Examples of useful silicone adhesives include the BioPSA® series (7-4400, 7-4500, and 7-4600 series) and the amine compatible (end-capped) BioPSA® series (7-4100, 7-4200, and 7-4300 series) manufactured by Dow Corning. In one or more embodiments, BioPSA® 4202 is used as the silicone adhesive. The silicone adhesive can be used in an amount in the range of about 20 to about 60 wt. %, including about 20 wt. %, about 25 wt. %, about 30 wt. %, about 35 wt. %, about 40 wt. %, about 45 wt. %, about 50 wt. %, about 55 wt. %, and about 60 wt. %. Suitable acrylate adhesive include polyacrylate adhesives such as pressure-sensitive adhesives based on an acrylate-vinylacetate polymer, such as, without limitation, DuroTak® 2054 and DuroTak® 2051. The acrylate adhesive can be used in an amount in the range of about 20 to about 60 wt %, including about 20 wt. %, about 25 wt. %, about 30 wt. %, about 35 wt. %, about 40 wt. %, about 45 wt. %, about 50 wt. %, about 55 wt. %, and about 60 wt. %.

In one or more embodiments, the silicone-type adhesive and the acrylate-type adhesive are present in a ratio range of about 1 to about 2.0 of silicone-type adhesive to acrylate type adhesive, including about 1, about 1.1, about 1.2, about 1.3, about 1.4, about 1.5, about 1.6, about 1.7, about 1.8, about 1.9, and about 2.0. In one or more specific embodiments, the silicone-type adhesive and the acrylate-type adhesive are present in a ratio range of about 1 to about 1.8 of silicone-type adhesive to acrylate type adhesive. In one or more very specific embodiments, the silicone-type adhesive and the acrylate-type adhesive are present in a ratio range of about 1.2 to about 1.4 of silicone-type adhesive to acrylate type adhesive. Without intending to be bound by theory, it is thought that the stable (i.e. no crystallization of buprenorphine is observed) combination of silicone and acrylate along with a solubilizer and a permeation enhancer is co-miscible. By changing the ratio of silicone to acrylate, a high/desired skin flux with minimized loading of buprenorphine may be achieved. The unexpected finding is adhesive is crucial/critical because it defines the stability and flux of the transdermal delivery system. Only when the transdermal system is prepared having the silicone-type adhesive and the acrylate-type adhesive present in a ratio range of about 1 to about 2.0, specifically about 1 to about 1.8, and more specifically about 1.2 to about 1.4, can a transdermal delivery system with desired flux and with high stability be prepared.

The pressure sensitive adhesive comprising a silicone-type adhesive blended with an acrylate-type adhesive may be present in an amount that generally ranges from about 50 to about75 wt. %, and in some embodiments, about 60 to about 70 wt. %, based on the total weight of the adhesive matrix.

In some embodiments, a tackifying agent is incorporated into the pressure-sensitive adhesive to improve the adhesive characteristics of the pressure-sensitive adhesive. Examples of suitable tackifying agents include polyterpenes, rosin derivatives, or silicone oils. In one or more embodiments, the tackifying agent is a silicone oil or fluid. In one or more embodiments, silicone fluids include high molecular weight polydimethylsiloxane, Dimethicone NF (Dow 360 Silicone Medical Fluid, 100 cSt and other viscosities). In specific embodiments, silicone fluid may be used in an amount of about 0% w/w to about 25%w/w and, more specifically, in a range of about 0% w/w to about 8.5% w/w.

In one or more embodiments, the pressure-sensitive adhesive is supplied and used in a solvent. Examples of suitable solvents include ethyl acetate, ethanol, isopropyl alcohol, heptane, volatile silicone fluids. The solids content is generally between about 40 and about 90%, including about 60 and about 80%.

In one or more embodiments, the solvent is evaporated before the final transdermal delivery system is provided. Without intending to be bound be theory, it is noted that the solvent is removed to avoid skin irritation

It is noted that the transdermal patch of one or more embodiments generally has a size that is about 20 to about 75%, including about 30 to about 60%, and about 40 to about 50% of the size of currently marketed transdermal patches, e.g. Butrans®. In one or more embodiments, the adhesive matrix generally has a size that is about 20 to about 75%, including about 30 to about 60%, and about 40 to about 50% of the size of the adhesive matrix of currently marketed patches, e.g. Butrans®. Then, a non-releasable backing layer is applied over the adhesive matrix layer. The result is a web-like structure comprised of a pressure-sensitive adhesive matrix layer, containing the drug, sandwiched between a non-releasable backing layer on one side and a disposable release liner on the other. The web can be cut into suitable sizes and shapes to produce pressure sensitive adhesive transdermal drug delivery patches.

In one or more embodiments, examples of a solubilizer that may be suitable for use in the adhesive matrix include a carboxylic acid. In one or more embodiment, the carboxylic acid is a C₃ to C₂₄ carboxylic acid including keto acids. In other embodiments, the carboxylic acid selected from the group consisting of levulinic acid, lauric acid, lactic acid, oleic acid, linoleic acid, linolenic acid, or mixtures thereof.

The solubilizer that may be present in an amount that generally ranges from about 3 to about 20 wt. %, and in some embodiments, about 3 to about 15 wt. %, or about 3 to about 10 wt. %, based on the total weight of the adhesive matrix.

In one or more embodiments, examples of a permeation enhancer that may be suitable for use in the adhesive matrix include a fatty acid ester. In certain embodiments, fatty acid esters include glycerol esters of fatty acids having 6 to 18 carbon atoms, where the glycerides may be monoglycerides, diglycerides, triglycerides, or combinations thereof. In specific embodiments, the fatty acid ester is selected from oleyl oleate, glyceryl monooleate, lauryl lactate, or mixtures thereof.

The permeation enhancer that may be present in an amount that generally ranges from about 3 to about 20 wt. %, and in some embodiments, about 3 to about 15 wt. %, or about 3 to about 10 wt. %, based on the total weight of the adhesive matrix.

In one or more embodiments, examples of a crystallization inhibitor that may be suitable for use in the adhesive matrix include different grades of cross-linked polyvinylpyrrolidone [cross-linked PVP], polyvinylpyrrolidone [povidone or PVP], hydroxypropyl methylcellulose [HPMC], and other cellulosic based materials or a mixture thereof.

In one or more embodiments, the transdermal patch comprises a cross-linked polyvinylpyrrolidone, which is added as a crystallization inhibitor and moisture absorber that allows maintenance of adhesion to the skin, especially during periods of heavy perspiration. Furthermore, the use of cross-linked polyvinylpyrrolidone does not diminish the buprenorphine skin flux.

The crystallization inhibitor that may be present in an amount that generally ranges from about 5 to about 30 wt. %, and in some embodiments, about 5 to about 20 wt. %, based on the total weight of the adhesive matrix.

A second aspect of the present invention is directed to method of treating pain in a patient comprising applying to the skin of a patient in need thereof, a transdermal delivery system comprising a buprenorphine base or a salt thereof, a solubilizer, a permeation enhancer, a crystallization inhibitor and a pressure sensitive adhesive including a silicone-type adhesive blended with an acrylate-type adhesive. In one or more embodiments, the transdermal delivery system is applied to the skin of the patient in need thereof for a time period in a range of 1 to 7 days. In other embodiments, the transdermal delivery system is applied to the skin of a patient in need thereof for a time period in a range of 1 to 3 days. In still further embodiments, the transdermal delivery system is applied to the skin of a patient in need thereof for a time period in a range of 1 to 180 hours, including a range of 1 to 168 hours, 1 to 144 hours, 1 to 120 hours, 1 to 96 hours, 1 to 72 hours, 1 to 48 hours, 1 to 24 hours, 24 to 168 hours, 24 to 144 hours, 24 to 120 hours, 24 to 96 hours, 24 to 72 hours, and 24 to 48 hours.

A third aspect of the present invention is directed to a method of preparing a transdermal system. In the one or more embodiments, the adhesive matrix is made in such a manner that components of the adhesive and their solvents are mixed with the drug and/or other substances and then coated on a suitable sheet, intended to function as a disposable liner, and the solvents are removed in a drying process. In one or more embodiments, the adhesive matrix is prepared by dispersing a crystallization inhibitor in a first solvent, e.g. ethyl acetate followed by slowly adding a second solvent, e.g. ethanol, to solubilize the crystallization inhibitor. Once the crystallization inhibitor is in solution, a solubilizer comprising a carboxylic acid and permeation enhancer comprising a fatty acid ester are added. Then, a pressure sensitive adhesive including a silicone-type adhesive blended with an acrylate-type adhesive is added. Finally, buprenorphine base or a salt thereof is added to achieve a homogenous mixture. The homogenous mixture is then coated on the release liner which is dried at about 80° C. to about 100° C. at about 500 rpm to about 1000 rpm and about 0.10 m/min to about 0.30 m/min speed.

The production of the transdermal delivery system requires a continuous coating and drying process to form the buprenorphine-containing adhesive matrix. Such coating is generally accomplished with a sufficiently sized roller coater and attached drying compartment. The buprenorphine-containing adhesive matrix mixture to be coated is usually prepared batch wise, and is then stored until the coater is ready to coat the mixture. The time between the preparation of the adhesive matrix mixture and the coating of the mixture in a normal production routine can be almost zero, if after mixing/homogenizing the mass will be transferred to the coating station and is coated directly, and may be as long as several days, e.g. four to six days, to store the adhesive matrix mixture during the time of a coating process interruption. Thus the adhesive matrix mixture must be sufficiently stable.

In one or more embodiments, the transdermal delivery system has the formulation of components appearing in Table 1. All values are % (w/w) on a dry basis.

TABLE 1
Composition % (w/w)

| Component | Range | Preferred |
|---|---|---|
| Buprenorphine | 3-15% | 5-10% |
| Silicone Adhesive | 20-60% | 36-41% |
| BIO-PSA ® 4202 | | |
| Acrylic Adhesive | 20-60% | 30% |
| Duro-Tak ® 387-2054 | | |
| Glyceryl Monooleate | 3-15% | 3% |
| Lauryl lactate | 3-15% | 7% |
| Levulinic acid | 3-10% | 7% |
| Polyvinylpyrrolidone | 5-20% | 10% |
| K-90 | | |

The invention is now described with reference to the following examples. Before describing several exemplary embodiments of the invention, it is to be understood that the invention is not limited to the details of construction or process steps set forth in the following description. The invention is capable of other embodiments and of being practiced or being carried out in various ways.

### EXAMPLES

### Example 1

Method of Manufacturing: Formulation 1 and Formulation 3 (described in Table 2, below) were prepared by dispersing PVP-K90 in ethyl acetate, followed by slowly adding ethanol to solubilize the PVP-K90. Levulinic acid and lauryl lactate were added, and the solution was stirred. Acrylate (DuroTak® 2054 and silicone adhesives (BIOPSA®-4202) were then added. Buprenorphine base was added to achieve a homogenous mixture. The mixture was coated onto a film of release liner Scotchpak® 9744, followed by drying at about 80° C. to about 95° C. at about 750 rpm to about 950 rpm and about 0.10 m/min to about 0.15 m/min speed. They are processing aids and they are not present in the final formulation as they evaporate during drying step.

### Example 2

Method of Manufacturing: Formulation 2 (described in Table 2, below) was prepared by dispersing PVP-K90 in ethyl acetate, followed by slowly adding ethanol to solubilize the PVP-K90. Levulinic acid and glycerol monooleate were added, and the solution was stirred. Acrylate (DuroTak® 2054 and silicone adhesives (BIOPSA®-4202) were then added. Buprenorphine base was added to achieve a homogenous mixture. The mixture was then coated onto a film of release liner Scotchpak® 9744 followed by drying at about 80° C. to about 95° C. at about 750 rpm to about 950 rpm and about 0.10 m/min to about 0.15 m/min speed.

Formulations 1-3 are described below in Table 2. Formulations 1-3 were prepared employing the Method of Manufacturing as described in Example 1 and Example 2 as described above. Formulation 1 was made using lauryl lactate as a permeation enhancer with 10% drug loading. Formulation 2 was made using glyceryl monooleate as a permeation enhancer with 10% drug loading. Formulation 3 was made using lauryl lactate as a permeation enhancer with 5% drug loading.

TABLE 2

| | Formulation 1 | Formulation 2 | Formulation 3 |
|---|---|---|---|
| Ingredients | (% of total) | (% of total) | (% of total) |
| Buprenorphine | 10 | 10 | 5 |
| BIO-PSA 4202 | 36 | 40 | 41 |
| DUROTAK 387- | 30 | 30 | 30 |
| 2054 | | | |
| Glyceryl Monooleate | — | 3 | — |
| Lauryl lactate | 7 | — | 7 |
| Levulinic acid | 7 | 7 | 7 |
| PVP K-90 | 10 | 10 | 10 |
| Ethanol* | 22.5 | 22.5 | 22.5 |
| Ethyl acetate* | 20 | 20 | 20 |
| Total | 100 | 100 | 100 |

*Processing aid/solvent - not included in total percentage.

### Example 4

Table 3 below depicts the physical parameters of Formulation 1 and the commercially available product Butrans®. There is a significant improvement in the adhesion of the Formulation 1 as compared to Butrans®. Formulation 1 has a single layer adhesive matrix system, and the peel adhesion is comparable to the peripheral adhesion layer in the Butrans® system. The tests demonstrate that Formulation 1 does not require an additional peripheral adhesion layer as present in Butrans® to achieve comparable peel adhesion. This improvement directly impacts the cost of manufacturing and the raw materials needed, while being more patient compliant.

TABLE 3

| | | Probe | |
|---|---|---|---|
| | | Tack (N) | Peel Adhesion (N) |
| Formulation 1 | | 3.208 | 9.789 |
| Butrans ® | Active adhesion | 2.138 | 3.839 |
| | layer | | |
| Butrans ® | Peripheral adhesion | 3.170 | 9.421 |
| | layer | | |

Probe Tack Test

Measurement of tack, reported as the maximum force (Newtons) required separating the bond between the adhesive and the probe, utilizes the Chem-Instrument PT-1000 and EZ Lab Software in accordance with ASTM D2979-01.

Peel Adhesion Test

This technique measures the force in Newtons required to peel a ½ inch wide strip from a Transdermal System from a Stainless Steel plate at a 180° peel angle using Chem-Instrument AR-1000 and EZ Lab software in accordance with ASTM D3330/D3330M. The reported peel force is the average peel value at 300 cm/minute over the plateau region of the force-time plot.

### Example 5

Formulation 2 was prepared using lauryl lactate as a permeation enhancer and 5% loading of buprenorphine. The formulation had comparable skin flux as compared to Butrans®, with half the amount of buprenorphine than in Butrans®. Developing a smaller transdermal patch with acceptable pharmacokinetic properties as compared to larger systems is difficult. Size directly impacts the amount of drug that can be loaded, the cost of manufacturing, and the raw materials used. Smaller patch size improves patient compliance by providing comfort to the patient.

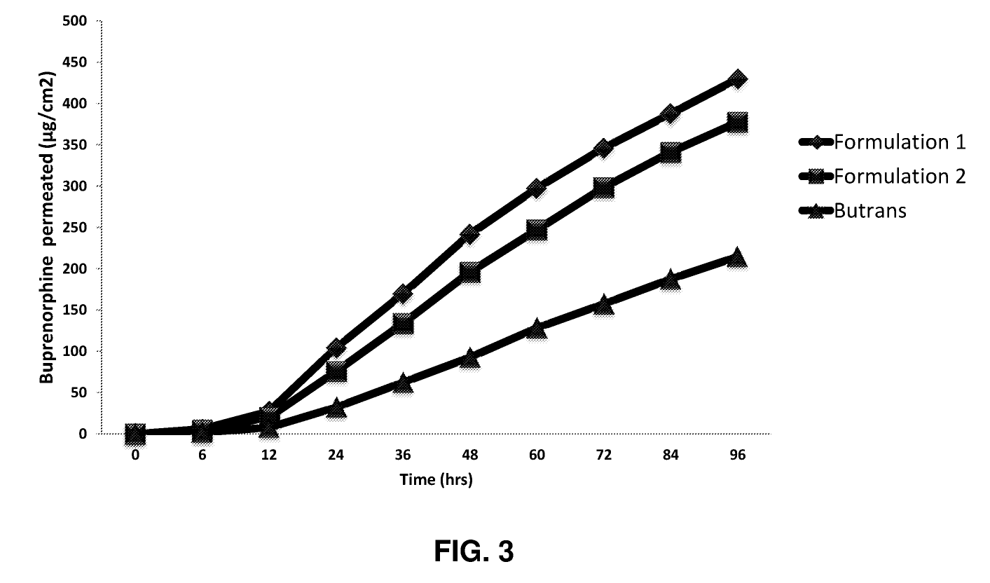

FIG. 3 shows the diffusion profile of Formulation 1 and Formulation 3 when tested against Butrans®. Formulation 1 and Formulation 3 have approximately 2 fold higher skin flux than the commercially marketed patch, sold under the trademark, Butrans®, while loading the same amount (10%) of buprenorphine in the patch. The data demonstrates that the patch size of Formulation 1 and Formulation 3 can effectively be reduced in half in order to deliver similar amount of buprenorphine as Butrans®.

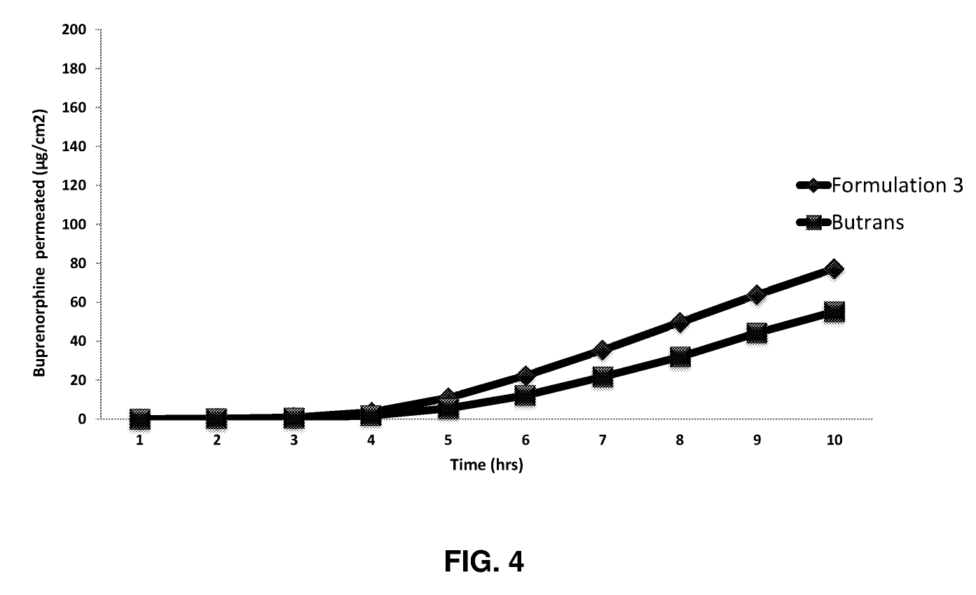

FIG. 4 shows the diffusion profile of Formulation 2 when tested against Butrans®. Formulation 2 has comparable skin flux when compared to Butrans®, but with half the amount of buprenorphine loaded on the patch, i.e., 5% drug loading for Formulation 2 versus 10% drug loading for Butrans®. The data demonstrates that drug loading can be cut in half when compared to the commercially available drug, resulting in less residual drug in the patch after use.

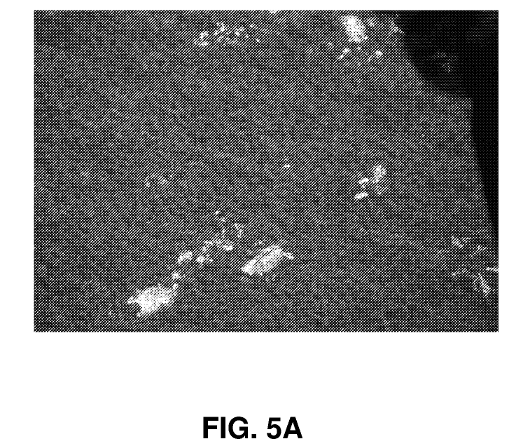

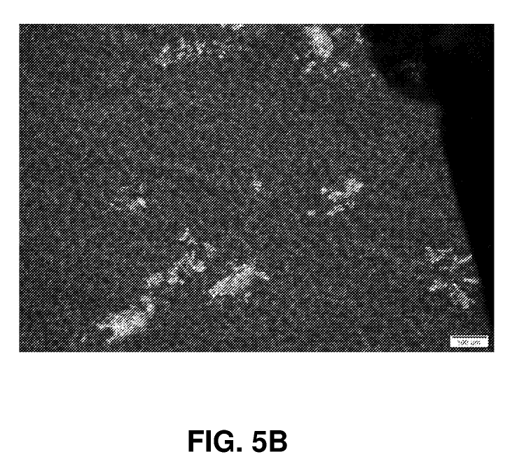

FIGS. 5A-5B shows the results of a crystallization study that was performed by seeding buprenorphine crystals in the patches of Formulation 1. After seeding the crystals, patches were analyzed for any new growth of crystals or increase in the size of seeded crystals under a polarized microscope. FIG. 5A shows the patch after the initial seeding. FIG. 5B shows the patch after 10 days. As illustrated in FIG. 5B, after 10 days, there was no increase in crystal growth and no additional crystals were observed for Formulation 1 which has 10% buprenorphine loading. FIG. 5 illustrates the stability of Formulation 1 at even 10% loading.

Thus, as shown in FIGS. 5A-5B, the stable (i.e. no crystallization observed) combination of a silicone-type adhesive and an acrylate-type adhesive, along with a solubilizer and a permeation enhancer is co-miscible. By changing the ratio of silicone-type adhesive to an acrylate-type adhesive, a high/desired skin flux with minimized loading of buprenorphine is achieved. The ratio of silicone to acrylate pressure sensitive adhesive is crucial/critical as it defines the stability and flux of the system. A system with desired flux and with high stability can be formulated using an appropriate ratio. The ratio of silicone to acrylate is in the range of 1.2-1.4. The unexpected finding is advantageous when using an expensive and controlled substance, e.g. buprenorphine, prone to abuse.

The use of the terms “a” and “an” and “the” and similar referents in the context of describing the materials and methods discussed herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the materials and methods and does not pose a limitation on the scope unless otherwise claimed. No language in the specification should be construed as indicating any non-claimed element as essential to the practice of the disclosed materials and methods.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A transdermal delivery system comprising:
a backing layer;
an adhesive matrix comprising buprenorphine or a salt thereof, a pressure sensitive adhesive including a silicone-type adhesive blended with an acrylate-type adhesive, a solubilizer, a permeation enhancer, and a crystallization inhibitor; and
a release liner.

2. The transdermal delivery system of claim 1, wherein the silicone-type adhesive is selected from pressure sensitive adhesives comprising: silicone polymer and resin, and the acrylate-type adhesive is an acrylate-vinylacetate polymer.

3. The transdermal delivery system of claim 1, wherein the solubilizer comprises a carboxylic acid and/or the permeation enhancer comprises a fatty acid ester.

4. The transdermal delivery system of claim 3, wherein the solubilizer is a C₃ to C₂₄ carboxylic acid or a C₃ to C₂₄ keto acid, or is selected from the group consisting of: levulinic acid, lauric acid, lactic acid, oleic acid, linoleic acid, and mixtures thereof.

5. (canceled)

6. (canceled)

7. The transdermal delivery system of claim 63, wherein the fatty acid ester is a glycerol ester of a C₆ to C₁₈ fatty acids; or a monoglyceride, diglyceride, triglyceride, or combinations thereof; or is selected from the group consisting of: oleyl oleate, glyceryl monooleate, lauryl lactate, and mixtures thereof.

8. (canceled)

9. (canceled)

10. The transdermal delivery system of claim 1, wherein the crystallization inhibitor is selected from one or more of the group consisting of: cross-linked polyvinylpyrrolidone, polyvinylpyrrolidone (PVP), or hydroxypropyl methylcellulose (HPMC), and other cellulosic based inhibitors.

11. The transdermal delivery system of claim 1, wherein the backing layer is impermeable to the buprenorphine and/or the backing layer comprises one or more of: films of: polyethylene, polyethylene terephthalate (PET), polypropylene, polyurethane, ethylene vinyl acetate (EVA), or polyamide; a metal foil; paper; paper coated with a polymeric material; a PVC foam; a woven fabric; and a non-woven fabric.

12. (canceled)

13. The transdermal delivery system of claim 1, wherein the silicone-type adhesive and the acrylate-type adhesive are present in a ratio range of about 1 to about 1.8 of silicone-type adhesive to acrylate type adhesive.

14. The transdermal delivery system of claim 1, wherein the release liner comprises one or more of: paper; coated paper; a plastic film; polyolefins comprising high density polyethylene (HDPE), low density polyethylene (LDPE), or polypropylene (PP) plastic resin; and fluoropolymer-coated films and/or the adhesive matrix further comprises a solvent selected from the group consisting of: ethanol, ethyl acetate, isopropyl alcohol, heptanes, and mixtures thereof.

15. (canceled)

16. The transdermal delivery system of claim 1, wherein the adhesive matrix comprises:
3 to 15% w/w buprenorphine,
20 to 60% w/w silicone adhesive,
20 to 60% w/w polyacrylate adhesive,
3 to 15% w/w cross-linked polyvinylpyrrolidone,
3 to 15% w/w carboxylic acid, and
3 to 15% w/w fatty acid; and
a release liner.

17. (canceled)

18. The transdermal delivery system of 16, wherein the adhesive matrix forms a single layer on the backing layer, and wherein there is no peripheral layer.

19. A method of relieving pain comprising: applying to skin of a patient in need thereof the transdermal delivery system of claim 1.

20. (canceled)

21. A method of preparing a transdermal delivery system, the method comprising:
dispersing a crystallization inhibitor in a first solvent to form a first solution;
adding a second solvent to the first solution to form a second solution;
adding a solubilizer and a permeation enhancer to the second solution to form a first mixture;
adding a pressure sensitive adhesive including a silicone-type adhesive blended with an acrylate-type adhesive to the first mixture to form a second mixture;
adding buprenorphine base or a salt thereof to the second mixture to form an adhesive matrix mixture; and
coating the adhesive matrix mixture on a backing layer.

22. The method of claim 21, wherein
the crystallization inhibitor is selected from one or more of the group consisting of: cross-linked polyvinylpyrrolidone, polyvinylpyrrolidone (PVP), or hydroxypropyl methylcellulose (HPMC), and other cellulosic based inhibitors, and/or
the first solvent and the second solvent are selected from the group consisting of: ethanol, ethyl acetate isopropyl alcohol, heptanes, and mixtures thereof; and/or
the solubilizer comprises a carboxylic acid; and/or
the permeation enhancer comprises a fatty acid ester, and/or
the backing layer comprises one or more of: films of: polyethylene, polyethylene terephthalate (PET), polypropylene, polyurethane, ethylene vinyl acetate (EVA), polyamide; a metal foil; paper; paper coated with a polymeric material; a PVC foam, a woven fabric, or a non-woven fabric.

23. (canceled)

24. (canceled)

25. The method of claim 21, wherein the solubilizer is a C₃ to C₂₄ carboxylic acid or a C₃ to C₂₄ keto acid; or is selected from the group consisting of: levulinic acid, lauric acid, lactic acid, oleic acid, and mixtures thereof.

26. (canceled)

27. (canceled)

28. The method of claim 21, wherein the permeation enhancer is a glycerol ester of a C₆ to C₁₈ fatty acid; or is a monoglyceride, diglyceride, triglyceride, or combinations thereof; or is selected from the group consisting of: oleyl oleate, glyceryl monooleate, lauryl lactate, and mixtures thereof.

29. (canceled)

30. (canceled)

31. (canceled)

32. The method of claim 21, wherein the silicone-type adhesive and the acrylate-type adhesive are present in a ratio range of about 1 to about 1.8 of silicone-type adhesive to acrylate type adhesive.

33. The method of claim 21, wherein the silicone-type adhesive is selected from the pressure sensitive adhesives comprising: silicone polymer and resin, and the acrylate-type adhesive is an acrylate-vinylacetate polymer.

34. An adhesive matrix comprising:
buprenorphine or a salt thereof;
a pressure sensitive adhesive including a silicone-type adhesive blended with an acrylate-type adhesive;
a solubilizer;
a permeation enhancer;
and a crystallization inhibitor.

35. The adhesive matrix of claim 34, wherein the adhesive matrix is formulated into a single layer on a backing layer and wherein there is no peripheral layer.
